(19) United States
(12) Patent Application Publication
(10) Pub. No.: US 2022/0347087 A1
(43) Pub. Date: Nov. 3, 2022

(54) HAIR TREATMENT PRODUCT AND METHOD OF MANUFACTURE AND USE

(71) Applicant: Shalita Grant, Hollywood, CA (US)

(72) Inventor: Shalita Grant, Hollywood, CA (US)

(21) Appl. No.: 17/244,607

(22) Filed: Apr. 29, 2021

(51) Int. Cl. A61K 8/98; A61K 8/73; A61K 8/9794; A61K 8/9789; A61K 8/92; A61K 8/34; A61Q 5/00; A61K 8/06

(52) U.S. Cl. CPC A61K 8/988; A61K 8/73; A61K 8/9794; A61K 8/9789; A61K 8/922; A61K 8/342; A61Q 5/00; A61K 8/06; A61K 2800/591; A61K 2800/805

(57) ABSTRACT

A hair treatment product is provided including Xanthan Gum in the amount of 0.5%-2.5% by volume, water in the amount of 20%-25% by volume, Henna powder in the amount of 25%-35% by volume, coffee grounds in the amount of 1%-3% by volume, Fenugreek in the amount of 1%-4% by volume, Aloe Vera juice in the amount of 8%-12% by volume, olive oil in the amount of 8%-13% by volume, honey in the amount of 15%-25% by volume, Saliguard PG in the amount of 0.5%-1.5% by volume, and peppermint oil in the amount of 0.5%-1.5% by volume. The hair treatment product is made by providing a first mixture including the Xanthan Gum and the water, providing a second mixture including the Henna powder, the coffee grounds, the Fenugreek, and the Aloe Vera juice, and providing a third mixture comprising the olive oil, the honey, the Saliguard PG, and the peppermint oil. The second mixture and the third mixtures are combined and mixed until homogeneous to form a fourth mixture. The fourth mixture and the first mixture are then combined and mixed until homogenous to form the hair treatment product. The method of applying the hair treatment product to a user's hair also is provided.

### COPYRIGHT STATEMENT

This patent document contains material subject to copyright protection. The copyright owner has no objection to the reproduction of this patent document or any related materials in the files of the United States Patent and Trademark Office, but otherwise reserves all copyrights whatsoever.

### FIELD OF THE INVENTION

This invention relates to hair treatment products, including hair treatment products for Type 4 hair.

### BACKGROUND

As is known in the art, the Andre Walker Hair Typing System is a system that classifies different types of hair into four main categories. The four main hair classification types include: 1) Straight, 2) Wavy, 3) Curly, and 4) Kinky. Each type is further classified into sub-types, with Type 4 hair including the sub-types: 4a (defined coil), 4b (z coil), and 4c (tight coil).

Type 4 hair is widely recognized as difficult to care for and is oftentimes dry (due to low porosity), brittle, and prone to breakage. Type 4 hair also sheds easily, tangles easily, is prone to matting, and is easily prone to heat damage. People with Type 4 hair also frequently experience hair shrinkage, have trouble retaining hair growth (experience thinning), and suffer from sensitive scalps (making it painful to detangle their hair).

However, many products available on the market are not aimed at helping to manage the hair in its natural curly state, but instead, are designed to alter the hair (e.g., straighten the hair) using harsh chemicals. However, because Type 4 hair is inherently fragile, these treatments weaken the bonds of the hair strands causing them to break even more easily.

In addition, methods currently known, taught, and practiced for managing Type 4 hair include shampooing the hair first and then attempting to detangle the hair after shampooing (often using detangling tools such as specialized brushes and/or combs). However, this technique often leads to even worse tangles and knots.

As described herein, the inventor has discovered a technique for using Henna mixed with other specialized ingredients as part of a novel product and method to manage Type 4 hair. Surprisingly however, because Henna is typically used as a hair dye product, cosmetology schools specifically warn stylists to not use Henna as it is known to be incompatible with bleached and chemically-treated hair. However, the specific product described herein, and the steps taken to manufacturer and apply the product to Type 4 hair, has been proven to be a novel solution to the problems described above.

Accordingly, there is a need for a method of manufacturer and use for a hair product that solves the problems related to managing Type 4 hair.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIGS. 1-5 show flowcharts depicting actions taken to manufacture a hair product according to exemplary embodiments hereof;

FIG. 6 shows a flowchart depicting actions taken to prepare a user's hair for the application of the hair product according to exemplary embodiments hereof; and

FIG. 7 shows a flowchart depicting actions taken to apply the hair product to a user's hair according to exemplary embodiments hereof.

### DETAILED DESCRIPTION OF EXEMPLARY EMBODIMENTS

In general, the presently disclosed hair treatment product and method relates to a hair treatment product, its process of manufacture, and its process of application and use. In some embodiments, the hair treatment product is designed for use with Type 4 hair.

For the purposes of this specification, the term “user” will mean the person receiving the hair treatment, and the term “provider” will mean the person providing the treatment to the user. The user and provider may typically be different people, but in some embodiments, it is understood that the user and the provider may be one in the same.

For the purposes of this specification, the constituents, and method(s) of manufacturing the hair treatment product will be described first. The method(s) of application of the hair treatment product will be described second. As described herein, the product includes a unique and novel combination of ingredients prepared using a unique and novel method. In addition, the acts taken to prepare a user's hair to receive the product, and the acts taken to apply the product to the user's hair also are unique and novel.

In some embodiments, the product 10 includes a restorative Henna mud masque that provides the following benefits (without limitation): (i) improved curl definition, (ii) noticeably shinier hair, (iii) noticeably softer hair, (iv) decreased shedding, (v) balanced porosity (improving the hair's ability to absorb and retain moister), (vi) healthy and weighted Type 4 curls, (vii) thicker hair, (viii) reduced dandruff, (ix) restoration of previously damaged hair, (x) other benefits, and any combinations thereof.

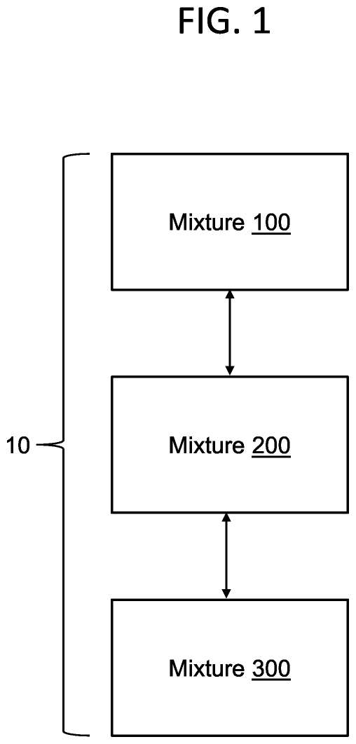

In some embodiments as shown in FIG. 1, the hair treatment product 10 includes a plurality of elements (ingredients) that may be combined using one or more methods as described herein. For example, in some embodiments the product 10 may include a first mixture 100 of elements, a second mixture 200 of elements, and a third mixture 300 of elements, that combined form the hair treatment product 10. In some embodiments, the product 10 includes an emulsion of at least some of these elements that is applied to a user's hair to provide the above benefits. As an emulsion, the product 10 is able to sustain and deliver each element and each combination of elements to the user's hair with higher efficiency.

Table 1 shows the elements included in each mixture 100, 200, 300 according to some exemplary embodiments. The table also shows each element's chemical trade name, International Nomenclature Cosmetic Ingredient name (INCI), percentage (%) of the total volume, Chemical Abstracts Service number (CAS), and European Inventory of Existing Commercial Chemical Substances (EINECS) number.

In some embodiments, each element's percentage (%) of the total volume may be varied by +/−20%.

TABLE 1

| | | Chemical | | % of | | |
|---|---|---|---|---|---|---|
| | | Trade | | Total | | |
| Element | Mixture | Name | INCI | Product | CAS | EINECS |
| 1 | 100 | Water | Water (Aqua) | 21.40 | 231-791-2 | 7732-18-5 |
| 2 | 100 | Xanthan Gum | Xanthan Gum | 1.50 | 11138-66-2 | 234-394-2 |
| 3 | 200 | Henna Color | Henna powder | 30.00 | 83-72-7/ | 201-496-3/ |
| | | (Dark Brown) | | | 84988-66-9 | 284-854-1 |
| 4 | 200 | Coffee ground | Coffee Arabica | 2.00 | 84650-00-0 | 283-481-1 |
| | | (fine) | (Coffee) Seed | | | |
| | | | Extract | | | |
| 5 | 200 | Fenugreek | Trigonella | 2.50 | 84625-40-1 | 283-415-1 |
| | | | Foenum- | | | |
| | | | Graecum | | | |
| | | | (Fenugreek) | | | |
| | | | Seed Extract | | | |
| 6 | 200 | Aloe Vera | Aloe | 10.00 | 8001-25-0 | 232-277-0 |
| | | Juice | Barbadensis | | | |
| | | | Leaf Juice | | | |
| 7 | 300 | Extra Virgin | Olea Europaea | 10.80 | 8001-25-0 | 232-277-0 |
| | | Olive Oil | (Olive) Fruit Oil | | | |
| 8 | 300 | Honey | Mel | 19.80 | 91052-92-5/ | 293-255-4 |
| | | | | | 8026-66-8 | |
| 9 | 300 | Saliguard PG | Caprylyl Glycol (and) | 1.00 | 1117-86- | 90063-97-1/ |
| | | | Phenoxyethanol | | 8/122-99-6 | 204-589-7 |
| 10 | 300 | Peppermint Oil | Mentha arvensis | 1.00 | 90063-97-1 | 290-058-5 |
| | | | (Peppermint) Leaf Oil | | | |

It is understood that Table 1 is meant to demonstrate the elements included in some embodiments of the product 10 and is not limiting. It also is understood that the product 10 may not include all of the elements listed, and/or may include additional elements not listed.

### Benefits of Various Elements

As is known in the art, the standard process for applying henna to a user's hair it to mix henna powder with water and to apply the solution to the hair directly. However, as is known, this process is very drying to the hair, especially for Type 4 hair. In addition, standard henna is orange such that the application of the henna to the user's hair causes the user's hair to be colored orange. To solve these problems, the hair treatment product 10 includes the plurality of elements 1-10 shown in Table 1, that are combined into a novel henna emulsion formula 10 using a novel method. In this way, the treatment 10 provides the benefits of the henna and other elements while counteracting the disadvantages of the henna itself. In addition, the Henna 3 included in the treatment 10 is premixed to provide a brown and/or black hue thereby mitigating the coloring of the hair orange. In other embodiments, the Henna 3 is premixed to provide a blond hue.

In some embodiments, the Henna (element 3 in Table 1) includes Cassia Obovata and/or other types of Henna. The Henna 3 , as included in the product 10 in the amounts specified and in combination with the other elements, penetrates the strands of hair and binds to the hair's keratin thereby strengthening the hair strands. The dye deposits of the Henna 3 fill the gaps within the hair shaft cuticles giving the hair added weight and strength. This in turn restructures the hair, enhances its shine, volumizes the hair, enables the hair to retain moister, and reveals and releases the natural curl pattern of the hair.

In some embodiments, the Henna 3 also reduces shedding and improves irritating scalp conditions such as inflammation and itching.

In some embodiments, the Henna 3 provides the benefits without adding color to the hair. In other embodiments, the Henna 3 provides the benefits while providing sufficient color deposits to adequately color gray hair.

In some embodiments, the Fenugreek (element 5) strengthens the hair strands, reduces shedding, and helps to treat poor scalp conditions.

The coffee 4, as included in the product 10 in the amounts specified and in combination with the other elements, helps to suppress the negative effects of Dihydrotestosterone (DHT). As is known in the art, DHT is a human androgen linked to hair loss. The coffee 4 includes caffeine that stimulates the hair roots so that hair follicles in an anagen growth phase are stimulated to grow stronger, thereby increasing the hair's stamina and endurance. In addition, the antioxidants in the caffeine reduce cell damage, stimulate healthy cell production, strengthens the hair shafts, and reduces trans-epidermal water loss (TDWL or TWL).

In addition, caffeine within the coffee 4 stimulates the proliferation of keratinocytes (the major protein components of hair) within the hair matrix where the hair shaft is produced (a critical part of the hair follicle anatomy). Caffeine also helps to inhibit phosphodiesterase (which plays a crucial role in alopecia) thereby protecting the user from catastrophic hair loss.

The Fenugreek 5, as included in the product 10 in the amounts specified and in combination with the other elements, includes antibacterial, antifungal, and anti-yeast properties that have been shown to reduce scalp conditions such as dandruff.

In addition, the antioxidant and anti-inflammatory properties of Fenugreek 5 reduce inflammation within the hair follicles thereby boosting the overall health of the user's scalp. This reduction in inflammation also reduces hair shedding and other types of hair loss.

Fenugreek 5 also is a rich source of vitamins A, K & C, folic acid, potassium, calcium, iron, and protein, which are essential nutrients for hair growth and for the repair of damaged (e.g., damage by heat tools and/or coloring) and dry hair. These elements also promote a healthy scalp environment that contributes to healthy hair growth.

The Aloe Vera juice 6, as included in the product 10 in the amounts specified and in combination with the other elements, includes fatty acids that help to resolve scalp inflammation caused by dandruff. In addition, the Aloe Vera 6 cleanses the hair shafts by stripping extra sebum (oil) and other residue left on the hair by other hair products.

The Extra Virgin Olive Oil 7, as included in the product 10 in the amounts specified and in combination with the other elements, includes vitamin E that acts as an antioxidant while promoting healthy hair growth. The Oil 7 also helps to rehydrate the hair by penetrating the hair shafts and sealing in moister. The antioxidants and nutrients in the Oil 7 also help to prevent different types of hair loss. Extra Virgin Olive Oil 7 also acts as a suffocating agent to head lice thereby being an effective treatment for this condition.

Other benefits provided by the product 10 and the combination(s) of elements 1-10 provided thereby are described herein.

### Preparation of the Mixtures 100, 200, 300

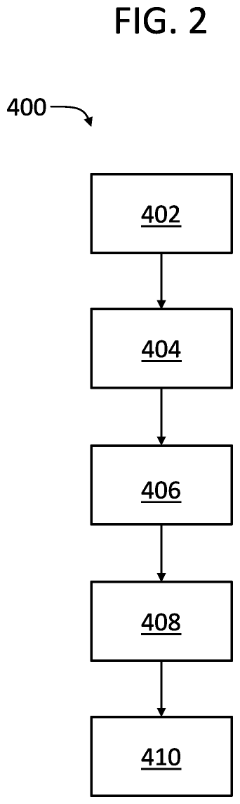

In some embodiments, as shown in FIGS. 2-4, the mixtures 100, 200, 300 may be formed by taking the following actions.

In some embodiments as shown in FIG. 2, the mixture 100 may be formed by performing the following actions 400:

402: Provide the water (element 1) and the Xanthan Gum (element 2);

404: Bring the elements 1, 2 to 25° C. ±10° (or at least to a temperature <50⁰);

406: Combine the elements 1, 2;

408: Blend the elements 1, 2 using a mixer (e.g., an electric mixer);

410: Stop blending the elements 1, 2 once thickened.

It is understood that the actions 400 described above are meant for demonstration and are non-limiting, and that other actions may be taken in addition to those listed. It also is understood that not all of the actions need be taken and that the actions may be taken in different order (e.g., action 406 may be taken prior to action 404).

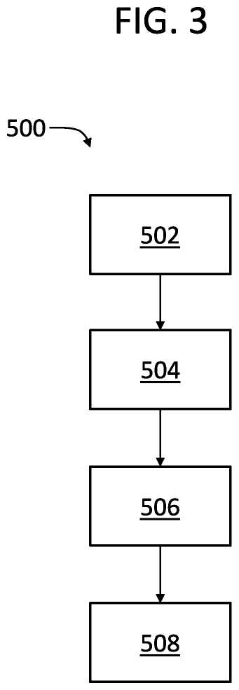

In some embodiments as shown in FIG.3, the mixture 200 may be formed by performing the following actions 500:

502: Provide the Henna color (element 3), the Coffee Grounds (element 4), the Fenugreek (element 5), and the Aloe Vera Juice (element 6);

504: Bring the elements 3, 4, 5, 6 to 25° C. ±10° (or at least to a temperature) <50° ;

506: Combine the elements 3, 4, 5, 6;

508: Mix the elements 3, 4, 5, 6 until homogeneous.

It is understood that the actions 500 described above are meant for demonstration and are non-limiting, and that other actions may be taken in addition to those listed. It also is understood that not all of the actions need be taken and that the actions may be taken in different order (e.g., action 506 may be taken prior to action 504).

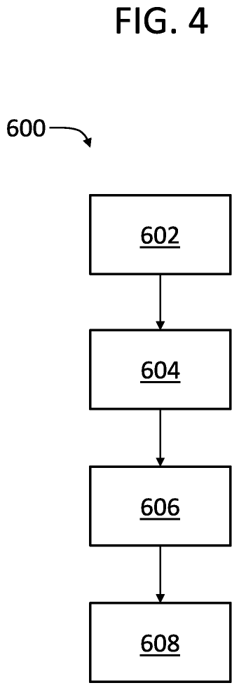

In some embodiments as shown in FIG. 4, the mixture 300 may be formed by performing the following actions 600:

602: Provide the Extra Virgin Olive Oil (element 7), the Honey (element 8), the Saliguard PG (element 9), and the Peppermint Oil (element 10);

604: Bring the elements 7, 8, 9 to 45° C. ±10° (or at least to a temperature <50°)

606: Bring element 10 to 45° C. ±10° (or at least to a temperature)<50°);

608: Combine the elements 7, 8, 9, 10.

It is understood that the actions 500 described above are meant for demonstration and are non-limiting, and that other actions may be taken in addition to those listed. It also is understood that not all of the actions need be taken and that the actions may be taken in different order.

### Forming the Hair Treatment Product 10

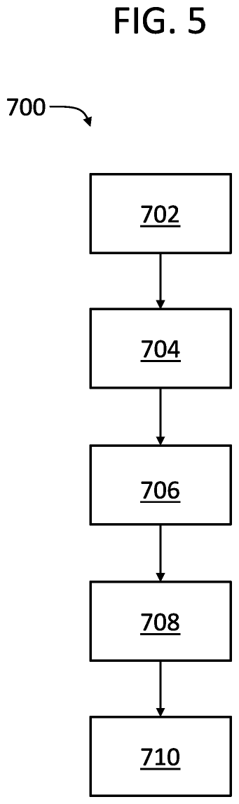

In some embodiments, as shown in FIG. 5, the product 10 may be formed by taking the following actions 700.

702: Perform actions 400, 500, and 600;

704: Combine mixture 300 with mixture 200;

706: Mix the combination from 704 until homogeneous;

708: Combine the mixture from 706 with mixture 100;

710: Mix the combination from 708 until homogeneous to form the hair treatment product 10.

It is understood that the actions 700 described above are meant for demonstration and are non-limiting, and that other actions may be taken in addition to those listed. It also is understood that not all of the actions need be taken and that the actions may be taken in different order.

In some embodiments, the resulting hair treatment product 10 is provided to a user and/or a provider in a container such as, without limitation, a jar, a bottle, a tube, and/or in other suitable containers. In some embodiments, the container may comprise glass and/or plastic, and may be generally transparent or may include a color (e.g., amber colored glass).

### Applying the Hair Treatment Product 10

This section will begin by describing the core actions taken during the application of the product 10. Additional details of certain actions will be described second.

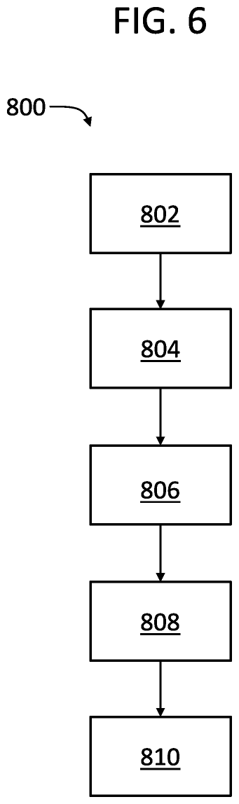

In some embodiments, as shown in FIG. 6, a user's hair may be prepared for the application of the product 10 with the provider taking the following actions 800.

802: Wet the user's hair with water (preferably soak the hair completely);

804: Section off the hair into one or more sections (e.g., into six sections);

806: Apply a deep hair conditioner and olive oil into each section of hair (preferably one section at a time);

808: Detangle each section of hair using a hair detangling instrument (e.g., brush, comb, etc.);

810: Apply shampoo to each section of hair, wash, and rinse.

In some embodiments, the actions 800 taken specifically require that the hair be conditioned and detangled (e.g., actions 802-808) prior to shampooed (e.g., action 810). This sequence of actions properly prepares the hair for the application of the product 10 thereby providing the benefits of the product 10 as described herein.

It is understood that the actions 800 described above are meant for demonstration and are non-limiting, and that other actions may be taken in addition to those listed. It also is understood that not all of the actions need be taken and that the actions may be taken in different order.

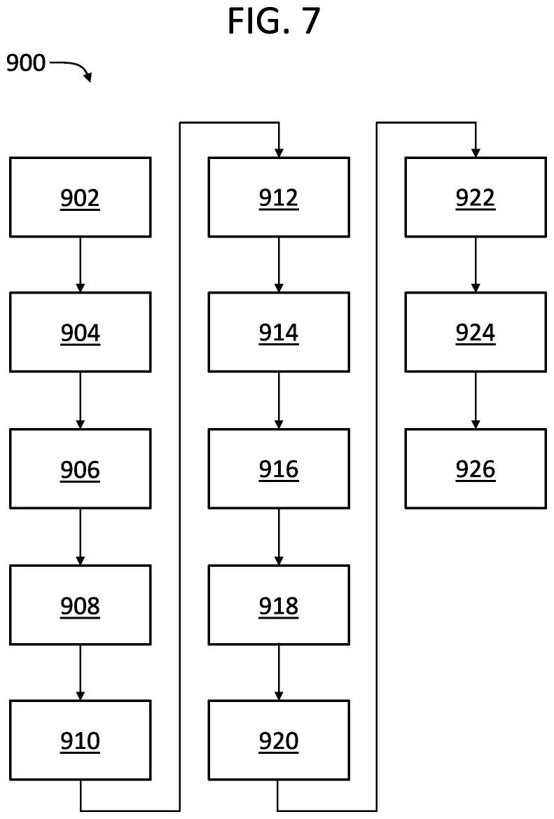

In some embodiments, as shown in FIG. 7, the provider may apply the product 10 to the user's hair by taking the following actions 900. It is preferred that actions 800 be performed first, and that actions 900 be performed after actions 800.

902: Apply coconut oil to the user's ears and skin in the areas adjacent the user's hairline. This will help to prevent these areas from becoming stained or otherwise discolored during the treatment;

904: Place the product 10 into a container (e.g., a bowl) and add water (preferably about ½ cup to 2 cups of warm water per product package;

906: Stir the mixture until it is homogeneous with a yogurt-like consistency. The resulting mixture is a Henna mud masque and, in some embodiments, may be clay-like in texture. The mixture also is an emulsion.

908: Starting at the nape of the user's neck, apply the mixture 806 by gently squeezing it into the user's hair starting at the ends of the hair. It may be preferable for the provider to wear gloves;

910: Smooth the mixture into the user's scalp;

912: Continue acts 908 and 910 until the user's entire head of hair is thoroughly covered with the mixture. Note: It is preferable that the provider does not allow the user's hair to dry during this application, and may continually wet the user's hair as necessary (e.g., using a spray bottle);

914: Cover the treated hair with a plastic cap. A paper towel (or similar) may be placed on top of the cap, especially in the areas of the user's nape and behind his/her ears. The cap and towel may then be tightly secured in place using plastic wrap extending around the plastic cap area. The plastic wrap may then be covered using a scarf or other type of headwear (e.g., headwear meant for sleeping);

916: Leave the treatment and the cap on the hair for approximately 2-10 hours;

918: Rinse the hair thoroughly;

920: Shampoo the hair to remove any henna and/of coffee debris;

922: Apply a deep conditioner to the hair and allow to sit for about 20 minutes. The provider should not detangle the hair with the conditioner on the hair at this stage.

924: Rinse the deep condition from the hair; and

926: Style as desired. Castor oil (or other types of sealing oils) also may be applied to the hair prior to styling for users intending to wear his/her hair curly, however, for those intending to flat iron his/her hair, it may be preferable that castor oil not be used.

It is understood that the actions 900 described above are meant for demonstration and are non-limiting, and that other actions may be taken in addition to those listed. It also is understood that not all of the actions need be taken and that the actions may be taken in different order.

### Additional Details

In some embodiments, the following details may apply to the actions described above.

Regarding action 804, the hair may be parted down the center and each side section may then be sectioned into three parts. It may be preferable for a first part on each side to be placed in front of the user's ear, and a second part to be placed behind the user's ear. Duckbill clips may be used on the ends of the hair to keep the sections separated. When creating the sections, it is preferred that any tangled clumps of hair are not detangled at this stage, as attempting to pull any hair apart from knots at this time may increase the probability of hair breakage.

Regarding action 806, it may be preferable for the provider to gently squeeze the conditioner into the user's wet hair going from root to end. A dollop or at least enough to coat each section of hair with conditioner should be used. It is preferable that the provider does not drag his/her fingers, rake-like, through the user's hair during this process. The olive oil may be sprayed onto each section of hair using a spray bottle. Depending on the thickness and length of hair, enough olive oil should be used to make each section feel slick but not soggy. This pre-shampoo mixture (also referred to as “pre-poo”) provides the hair with adequate slip (a measure of how lubricious the product makes the hair), making the hair easier to detangle while helping to prevent unnecessary hair breakage, rips, tears, and split ends (all important factors for the retention of Type 4 hair when detangling). In addition, during this process, if any section of hair becomes dry, the hair should be wetted (e.g., using a spray bottle) before the deep conditioner and/or olive oil is applied. This helps to ensure that the cuticles of the hair are open.

Regarding action 808, it may be preferable that the provider start the detangling with the first section of hair that received the pre-conditioning mixture. For thick sections of hair, the process may include splitting the section of hair in half and starting with a section closest to the hairline. The provider may hold the hair using one hand while dragging the detangling instrument through the hair using the other hand, starting from the ends. After each stroke with the brush, the provider may smooth the brushed hair using his/her thumb, forefinger, and middle finger of his/her free hand. With each brushstroke, the provider may work closer and closer to the root and scalp. If the provider encounters a knot, the provider should brush the section of hair under the knot to work out and detangle the section.

During this detangling process, the provider may encounter one or more of the following hair knot types:

1. Single Strand Knots (also referred to a “Fairy Knot”): These knots form when a single strand of hair becomes tangled and knotted on itself.

2. Matted hair knots: These knots form when shed hair gets trapped around hair still attached to the scalp.

3. Clumped curls: These knots feel stiff but can usually be worked out by brushing underneath the clumped section.

When a provider may encounter these or other types of hair knots, the provider may place down the brush and assess the knot. He/she may then gently pull hair attached to the scalp away from the knotted strands. This may be facilitated by the fact that the hair contains the pre-shampoo mixture. Once the provider has released all or most of the willing strands of hair, he/she may go back to using the detangling brush working from the ends and smoothing gently behind. This process may be repeated until the knot has been removed.

In some embodiments, the product 10 is designed for home use while in other embodiments the product 10 is designed for use at a salon. In other embodiments, the product 10 is designed for use at home and/or in a salon.

It is understood that any aspects or elements of any embodiment described herein or otherwise may be combined with any other aspect or element of any other embodiment to form one or more additional embodiments all of which are within the scope of the product 10, its method of manufacture, and its method of application.

Those of ordinary skill in the art will appreciate and understand, upon reading this description, that embodiments hereof may provide different and/or other advantages, and that not all embodiments or implementations need have all advantages.

Where a process is described herein, those of ordinary skill in the art will appreciate that the process may operate without any user intervention. In another embodiment, the process includes some human intervention (e.g., a step is performed by or with the assistance of a human).

As used herein, including in the claims, the phrase “at least some” means “one or more,” and includes the case of only one. Thus, e.g., the phrase “at least some ABCs” means “one or more ABCs”, and includes the case of only one ABC.

As used herein, including in the claims, term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

As used in this description, the term “portion” means some or all. So, for example, “A portion of X” may include some of “X” or all of “X”. In the context of a conversation, the term “portion” means some or all of the conversation.

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

It should be appreciated that the words “first,” “second,” and so on, in the description and claims, are used to distinguish or identify, and not to show a serial or numerical limitation. Similarly, letter labels (e.g., “(A)”, “(B)”, “(C)”, and so on, or “(a)”, “(b)”, and so on) and/or numbers (e.g., “(i)”, “(ii)”, and so on) are used to assist in readability and to help distinguish and/or identify, and are not intended to be otherwise limiting or to impose or imply any serial or numerical limitations or orderings. Similarly, words such as “particular,” “specific,” “certain,” and “given,” in the description and claims, if used, are to distinguish or identify, and are not intended to be otherwise limiting.

As used herein, including in the claims, the terms “multiple” and “plurality” mean “two or more,” and include the case of “two.” Thus, e.g., the phrase “multiple ABCs,” means “two or more ABCs,” and includes “two ABCs.” Similarly, e.g., the phrase “multiple PQRs,” means “two or more PQRs,” and includes “two PQRs.”

The present invention also covers the exact terms, features, values and ranges, etc. in case these terms, features, values and ranges etc. are used in conjunction with terms such as about, around, generally, substantially, essentially, at least etc. (i.e., “about 3” or “approximately 3” shall also cover exactly 3 or “substantially constant” shall also cover exactly constant).

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components unless specifically so stated.

It will be appreciated that variations to the embodiments of the invention can be made while still falling within the scope of the invention. Alternative features serving the same, equivalent or similar purpose can replace features disclosed in the specification, unless stated otherwise. Thus, unless stated otherwise, each feature disclosed represents one example of a generic series of equivalent or similar features.

The present invention also covers the exact terms, features, values and ranges, etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

Use of exemplary language, such as “for instance”, “such as”, “for example” (“e.g.,”) and the like, is merely intended to better illustrate the invention and does not indicate a limitation on the scope of the invention unless specifically so claimed.

While the invention has been described in connection with what is presently considered to be the most practical and preferred embodiments, it is to be understood that the invention is not to be limited to the disclosed embodiment, but on the contrary, is intended to cover various modifications and equivalent arrangements included within the spirit and scope of the appended claims.

1. A method of making a hair treatment product, the method comprising:
(A) providing a first mixture comprising Xanthan Gum in the amount of 0.5%-2.5% by volume, and water in the amount of 20%-25% by volume;
(B) providing a second mixture comprising Henna powder in the amount of 25%-35% by volume, coffee grounds in the amount of 1%-3% by volume, Fenugreek in the amount of 1%-4% by volume, and Aloe Vera juice in the amount of 8%-12% by volume;
(C) providing a third mixture comprising Olive Oil in the amount of 8%-13% by volume, Honey in the amount of 15%-25% by volume, Saliguard PG in the amount of 0.5%-1.5% by volume, and Peppermint Oil in the amount of 0.5%-1.5% by volume;
(D) using a mixing device, mix the second mixture and the third mixture together until homogeneous to form a fourth mixture;
(E) using a mixing device, mix the fourth mixture with the first mixture until homogeneous.

2. The method of claim 1 wherein the first mixture is provided at 25° C. ±10° C.

3. The method of claim 1 wherein the second mixture is provided at 25° C. ±10° C.

4. The method of claim 1 wherein the Olive Oil, the Honey, and the Saliguard PG in the third mixture are provided at 45° C. ±10° C., and the Peppermint Oil in the third mixture is provided at 45° C. ±10° C.

5. The method of claim 1 wherein the result of (E) includes an emulsion.

6. A hair treatment product made of a composition comprising:
Xanthan Gum in the amount of 0.5%-2.5% by volume;
water in the amount of 20%-25% by volume;
Henna powder in the amount of 25%-35% by volume;
coffee grounds in the amount of 1%-3% by volume;
Fenugreek in the amount of 1%-4% by volume;
Aloe Vera juice in the amount of 8%-12% by volume;
Olive Oil in the amount of 8%-13% by volume;
Honey in the amount of 15%-25% by volume;
Saliguard PG in the amount of 0.5%-1.5% by volume; and
Peppermint Oil in the amount of 0.5%-1.5% by volume.

7. The hair treatment product of claim 6 wherein:
the Xanthan Gum is at 25° C. ±10° C.;
the water is at 25° C. ±10° C.;
the Henna powder is at 25° C. ±10° C.;
the coffee grounds are at 25° C. ±10° C.;
the Fenugreek is at 25° C. ±10° C.;
the Aloe Vera juice is at 25° C. ±10° C.;
the Olive Oil is at 45° C. ±10° C.;
the Honey is at 45° C. ±10° C.;
the Saliguard PG is at 45° C. ±10° C.; and
the Peppermint Oil is at 45° C. ±10° C.

8. A method of applying the hair treatment product of claim 6 to a user's hair, the method comprising:
(A) prepping the user's hair for the application of the hair treatment product;
(B) placing the hair treatment product into the user's hair;
(C) placing a cap over the hair that received the hair treatment product in (B);
(D) allowing the capped hair to sit for 2-10 hours;
(E) removing the cap; and
(F) rinsing the hair with water.

9. The method of claim 8 further comprising:
(G) shampooing the hair;
(H) applying a hair conditioner to the hair; and
(I) rinsing the hair with water.

10. The method of claim 8 wherein (A) further comprises:
(A)(1) wetting the user's hair with water;
(A)(2) sectioning the hair into at least two sections;
(A)(3) applying hair conditioner and/or olive oil to each section of hair;
(A)(4) detangling each section of hair;
(A)(5) shampooing each section of hair; and
(A)(6) rinsing each section of hair.

11. The method of claim 8 further comprising:
(B)(1) applying coconut oil to the user's ears and/or nape of the neck adjacent a back hairline.

12. The method of claim 8 further comprising:
(B)(2) adding warm water to the hair treatment product and mixing until homogeneous.

13. The method of claim 8 wherein the placing the hair treatment product into the user's hair in (B) includes squeezing the hair treatment product into the user's hair starting at one or more ends of the hair.

14. The method of claim 8 wherein the placing a cap over the hair that received the hair treatment in (C) includes placing a towel on the cap and securing the cap and the towel with plastic wrap.
